(19) United States
(12) Patent Application Publication
(10) Pub. No.: US 2024/0426776 A1
(43) Pub. Date: Dec. 26, 2024

(54) RAPID DIFFERENTIATION OF ANTIBIOTIC-SUSCEPTIBLE AND -RESISTANT BACTERIA THROUGH MEDIATED EXTRACELLULAR ELECTRON TRANSFER

(71) Applicant: WASHINGTON STATE UNIVERSITY, Pullman, WA (US)

(72) Inventors: Haluk BEYENAL, Pullman, WA (US); Abdelrhman MOHAMED, Pullman, WA (US); Douglas R. CALL, Pullman, WA (US); Gretchen TIBBITS, Pullman, WA (US)

(21) Appl. No.: 18/704,332

(22) PCT Filed: Oct. 28, 2022
(86) PCT No.: PCT/US2022/048267

§ 371 Date: Apr. 24, 2024

Related U.S. Application Data
(60) Provisional application No. 63/273,396, filed on Oct. 29, 2021.

(51) Int. Cl. G01N 27/416; C12M 1/00; C12M 1/34; G01N 27/327

(52) U.S. Cl. CPC G01N 27/416; C12M 29/06; C12M 41/46; G01N 27/3275

(57) ABSTRACT

A mediated extracellular electron transfer as a rapid and direct method to classify antibiotic-susceptible and -resistant bacteria is disclosed herein. Particularly, devices and methodologies to interpret antibiotic susceptibility index of a known bacteria strain or uncharacterized bacteria by treating it with antibiotics of different mechanisms or different concentrations of an antibiotic is provided. In an example methodology, the process includes a number of current control signal responses and a number of inoculated current response signals and thereafter analyzing the inoculated current response signals over a range of concentrations of up to about 24 times a breakpoint of the antibiotic with respect to current control signal responses to provide a susceptibility index assessment indicative of the susceptibility or resistance of the pathogen to the antibiotic.

### CROSS-REFERENCE TO RELATED APPLICATION

The present application claims under 35 U.S.C § 119, the priority benefit of U.S. Provisional Application No. 63/273,396, filed Oct. 29, 2021, entitled “RAPID DIFFERENTIATION OF ANTIBIOTIC-SUSCEPTIBLE AND-RESISTANT BACTERIA THROUGH MEDIATED EXTRACELLULAR ELECTRON TRANSFER.” which is incorporated herein by reference in its entirety.

### STATEMENT REGARDING FEDERALLY SPONSORED RESEARCH

This invention was made with government support under grant/contract no. CBET0954186 awarded by National Science Foundation. The government has certain rights in the invention.

### FIELD OF INVENTION

The embodiments herein relate to rapid and simultaneous identification of antibiotic-susceptibility and antibiotic-resistance of a bacteria. More particularly, the embodiments herein relate to devices and methods of interpreting antibiotic susceptibility having distinct mechanisms of action or interpreting differing concentrations of antibiotics utilized for treatment of a given known or uncharacterized bacteria or pathogen.

### BACKGROUND OF THE INVENTION

Antibiotic resistance is a leading health challenge on a global scale as antimicrobial-resistant bacteria have been associated with deaths. For this reason, antibiotic stewardship principles encourage susceptibility testing before antibiotic treatment to ensure that an antibiotic is well matched to a putative pathogen. For optimized treatment, having rapid access to information about pathogens involved and their antibiotic susceptibility and resistance profiles is necessary. Such information can assist in forming informed decisions about the most appropriate antibiotic to be utilized rather than relying on empirical guesses that could contribute to greater morbidity and mortality because of treatment failures, as well as selectively favoring increasingly drug-resistant pathogens.

In practice, however, the entire testing process from isolation to preparation and drug susceptibility testing takes 2-3 days, and consequently, antibiotics are often prescribed in the absence of better information. Faster methods are additionally beneficial, but the final stage of traditional antibiotic susceptibility testing relies on culturing bacteria with antibiotics on agar plates such as diffusion assays or in broth culture such as microdilution assays which require a minimum of 8 and up to 24 hours to complete. While the results of these tests are interpreted in the context of population growth i.e., zones on agar plates or optical density in broth culture, in practice, essentially these readouts are actually used as proxies for detecting bacterial respiration. Consequently, the assay results are delayed by the need to grow a sufficient population density over a period of hours to a day to classify bacterial strains as antibiotic-susceptible or -resistant.

As an alternative to this secondary measurement of respiration, electrochemical techniques can be used to directly detect bacterial respiration with a diversity of antibiotics. Through extracellular electron transfer, electrons are liberated via cellular respiration to the surface of inert electrodes resulting in a measurable flow of electrons i.e., electrical current. To date, however, these methods have not been systematically applied to classify different strains within the same species as antibiotic-resistant or -susceptible when challenged with antibiotics that work using varying mechanisms of action.

Background information on use of electrochemical techniques for measurement of cell respiration, is described and claimed in U.S. Pat. No. 4,209,586A entitled, “Method of testing the effectiveness of a growth inhibiting agent on a microorganism,” filed Jul. 14, 1977, to Hans G. Noller, including the following, “The changes in the redox potentials of cultures of a microorganism with and without a tested growth inhibiting agent are monitored during the phase of growth in which the redox potential is normally positive and the rate of potential change is approximately linear. Effective growth inhibiting agents produce a measurable decrease in the change of the redox potential to a more negative value within less than one hour . . . .”

Background information on use of electrochemical techniques for measurement of cell respiration, is described, “Rapid Electrochemical Monitoring of Bacterial Respiration for Gram-Positive and Gram-Negative Microbes: Potential Application in Antimicrobial Susceptibility Testing,” published in Analytical Chemistry (Volume 92, Issue 6, Pages 4266-4274 Feb. 12, 2020), including the following, “ . . . a rapid AST using electroanalysis with a 15 min assay time, called EAST, which is live-monitored by time-lapse microscopy video. The present work reports systematical electrochemical analysis and standardization of protocol for EAST measurement. The proposed EAST is successfully applied for Gram-positive Bacillus subtilis and Gram-negative Escherichia coli as model organisms to monitor bacterial concentration, decay kinetics in the presence of various antibiotics . . . . ”

Background information on use of electrochemical techniques in addition to electron transfer mediators for analysis of antimicrobial compounds, are described and claimed in U.S. Pat. No. 6,391,577B1 entitled, “Rapid Electrochemical Assay for Antibiotic and Cytotoxic Drug Susceptibility in Microorganisms,” filed May 21, 2002, to Mikkelsen et al, including the following, “The method for assessing susceptibility of a microorganism to an antibiotic or cytotoxic drug comprises adding a Suitable mediator or mediator mixture to a sample of the microorganism in the presence of the drug, and assessing variation of the microorganism's respiration rate overtime by electrochemical measurement of mediator consumption resulting from microorganism respiration. This is compared with variation of the respiration rate of another sample of the microorganism not exposed to the drug.”

Accordingly, a need exists for an improved antibiotic susceptibility test using electrochemical techniques for rapid results on different strains of pathogens and antibiotics with distinct mechanisms of action. The embodiments disclosed herein address such a need by way of a multi-electrode electrochemical device used with a growth medium and an electron transfer mediator to rapidly and simultaneously assess antibiotic susceptibility and resistance for multiple strains of different and important pathogen or uncharacterized pathogen, each tested with an antibiotic of either distinct mechanisms of action or different concentrations of the same antibiotic.

### BRIEF SUMMARY OF THE INVENTION

In a first aspect, the embodiments herein are directed to a multiplexed electrochemical drug susceptibility method, that includes: disposing a non-inoculated solution in one or more sample wells arranged in an array, wherein the non-inoculated solution in the one or more sample wells is configured with at least one of an electron transfer mediator, a growth culture, and a pathogen; measuring a multiplexed number of current control signal responses of the non-inoculated solution disposed in the one or more sample wells of the array; inoculating a set number of solutions in the one or more sample wells with an antibiotic, wherein the inoculating step includes a range of concentrations of up to about 24 times a breakpoint of the antibiotic; measuring a multiplexed number of inoculated current response signals from the inoculated set number of solutions in the one or more sample wells of the array; and analyzing with a computer control and data system/processor, the multiplexed number of current control signal responses and the multiplexed number of inoculated current response signals over the range of concentrations of up to about 24 times a breakpoint of the antibiotic to provide a susceptibility index assessment indicative of the susceptibility or resistance of the pathogen to the antibiotic.

In a second aspect, the embodiment herein are directed to a multiplexed electrochemical drug susceptibility system, that includes: an array of sample containers, wherein one or more sample containers of the array of sample containers are each configured with an interior volume configured to hold a solution that further comprises at least one of an electron transfer mediator, a drug, a growth culture, and a pathogen; a plurality of sets of two or more electrodes, wherein each of a set of the plurality of sets of two or more electrodes are coupled to the solution of the one or more sample containers; an interface configured to receive and isolate a multiplexed set of control signals and a multiplexed set of inoculated current signals from the plurality of sets of two or more electrodes; and a computer control and data system/processor coupled to the interface so as to interrogate the multiplexed set of control signals and the inoculated current signals for a susceptibility index assessment of the pathogen.

The embodiments herein thus enable a methodology as well as a system to distinguish between a pathogen's susceptibility or resistance to an antibiotic/drug in less than 90 min based on an electrical response provided over a wide dynamic range. In particular, the hereinafter deemed Rapid Electrochemical Assay for Detecting Antibiotic Susceptibility (READAS) technology provides susceptibility information in an often-multiplexed fashion sooner than is possible with current technologies, which provides a significant beneficial aspect for matching antibiotics to pathogens and limiting selection for antimicrobial-resistant bacteria.

### BRIEF DESCRIPTION OF THE DRAWINGS

FIG. 1A illustrates an example embodiment of an electrochemical system utilized for determination of an antibiotic susceptibility or resistance, as disclosed herein.

FIG. 1B shows a schematic of an electrochemical cell connected to a potentiostat.

FIG. 2A shows current responses of a bacterial culture of K. pneumoniae in the presence and absence of an electron transfer mediator.

FIG. 2B shows current responses of a bacterial culture of A. baumannii in the presence and absence of an electron transfer mediator.

FIG. 2C shows current responses of a bacterial culture of S. aureus in the presence and absence of an electron transfer mediator.

FIG. 2D shows current responses of a bacterial culture of E. coli in the presence and absence of an electron transfer mediator.

FIG. 3A shows cyclic voltammogram reading of E. coli in the presence and absence of an electron transfer mediator.

FIG. 3B shows the absorbance growth curves on increasing concentration of the electron transfer mediator with A. baumannii strain 78.

FIG. 3C shows the current response of A. baumannii inoculated at inoculum densities (OD600 0.0625 to 0.5) in presence of the electron transfer mediator.

FIG. 4A shows the current response after inoculating an electrochemical cell with A. baumannii susceptible strain in the presence and absence of an antibiotic.

FIG. 4B shows the current response after inoculating an electrochemical cell with A. baumannii resistant strain in the presence and absence of an antibiotic.

FIG. 5A shows the example raw current response after inoculating the electrochemical cell with 0.1±0.01 OD₆₀₀ A. baumannii susceptible strain.

FIG. 5B shows the example raw current response after inoculating the electrochemical cell with 0.1±0.01 OD₆₀₀ A. baumannii resistant strain.

FIG. 6 shows the decision methodology for determining antibiotic susceptibility and resistance.

FIG. 7A shows the antibiotic susceptibility index for a susceptible and a resistant bacteria strain of A. baumannii, each treated with and without an antibiotic (tobramycin and imipenem).

FIG. 7B shows the analysis time for a susceptible and a resistant bacteria strain of A. baumannii, each treated with and without an antibiotic (tobramycin and imipenem).

FIG. 8A shows the current response of a S. aureus strain treated with 4×, 6×, 8×, 10×, and 20× the breakpoint concentration of an antibiotic (oxacillin) and compared to control treatment.

FIG. 8B shows the current response of a S. aureus strain treated with 4× and 10× the breakpoint concentration of an antibiotic (tobramycin) and compared to control treatment.

FIG. 9A shows the antibiotic susceptibility index for a susceptible and a resistant bacteria strain of S. aureus, each treated with an antibiotic (tobramycin and oxacillin).

FIG. 9B shows the analysis time for a susceptible and a resistant bacteria strain of S. aureus, each treated with an antibiotic (tobramycin and oxacillin).

FIG. 10A shows the antibiotic susceptibility index for a susceptible and a resistant bacteria strain of E. coli, each treated with an antibiotic (tobramycin and imipenem).

FIG. 10B shows the analysis time for a susceptible and a resistant bacteria strain of E. coli, each treated with an antibiotic (tobramycin and imipenem).

FIG. 11A shows the current response of a K. pneumoniae strain treated with 6×, 10×, 16×, and 20× the breakpoint concentration of an antibiotic (imipenem) and compared to control treatment.

FIG. 11B shows the current response of a K. pneumoniae strain treated with 2×, 4×, 8×, 10× and 20× the breakpoint concentration of an antibiotic (ciprofloxacin) and compared to control treatment.

FIG. 12A shows the antibiotic susceptibility index for a susceptible and a resistant bacteria strain of K. pneumoniae, each treated with an antibiotic (ciprofloxacin and imipenem).

FIG. 12B shows the analysis time for a susceptible and a resistant bacteria strain of K. pneumoniae, each treated with an antibiotic (ciprofloxacin and imipenem).

FIG. 13A shows the current response and growth curves on the same time scale of A. baumannii susceptible (35) strain treated with 32 μg/mL tobramycin.

FIG. 13B shows the current response and growth curves on the same time scale of A. baumannii resistant (83) strain treated with 32 μg/mL tobramycin.

FIG. 14 shows the example embodiment of a multi-well reactor antibiotic susceptibility device, as disclosed herein.

### DETAILED DESCRIPTION OF THE INVENTION

In the description of the invention herein, it is understood that a word appearing in the singular encompasses its plural counterpart, and a word appearing in the plural encompasses its singular counterpart, unless implicitly or explicitly understood or stated otherwise. Furthermore, it is understood that for any given component or embodiment described herein, any of the possible candidates or alternatives listed for that component may generally be used individually or in combination with one another, unless implicitly or explicitly understood or stated otherwise. Moreover, it is to be appreciated that the figures, as shown herein, are not necessarily drawn to scale, wherein some of the elements may be drawn merely for clarity of the invention. Also, reference numerals may be repeated among the various figures to show corresponding or analogous elements. Additionally, it will be understood that any list of such candidates or alternatives is merely illustrative, not limiting, unless implicitly or explicitly understood or stated otherwise. In addition, unless otherwise indicated, numbers expressing quantities of ingredients, constituents, reaction conditions and so forth used in the specification and claims are to be understood as being modified by the term “about.”

Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

### General Description

It is to be appreciated that the disclosed embodiments herein are directed to an electrochemical method/system that preferably utilizes configured monitored multiple reactors/cells to assess for antibiotic susceptibility and/or resistance of particular pathogen strains, including gram-positive and gram-negative and/or previously characterized or uncharacterized-pathogens. Aspects of the embodiments include introduced mediators (electron transfer mediators) that interact with respiratory pathways of interrogated microorganisms. Such interactions enable the mediators to be reduced and thereafter guided/transported so as to be received by a working electrode that results in currents that correspond to microbe respiration processes (i.e., respiration-induced microbe current signals). Appreciably, such respiration-induced microbe current signals are indicative of the effectiveness of applied drugs (e.g., antibiotics) to the pathogens, i.e., the resistance or susceptibility of certain pathogens to antibiotics.

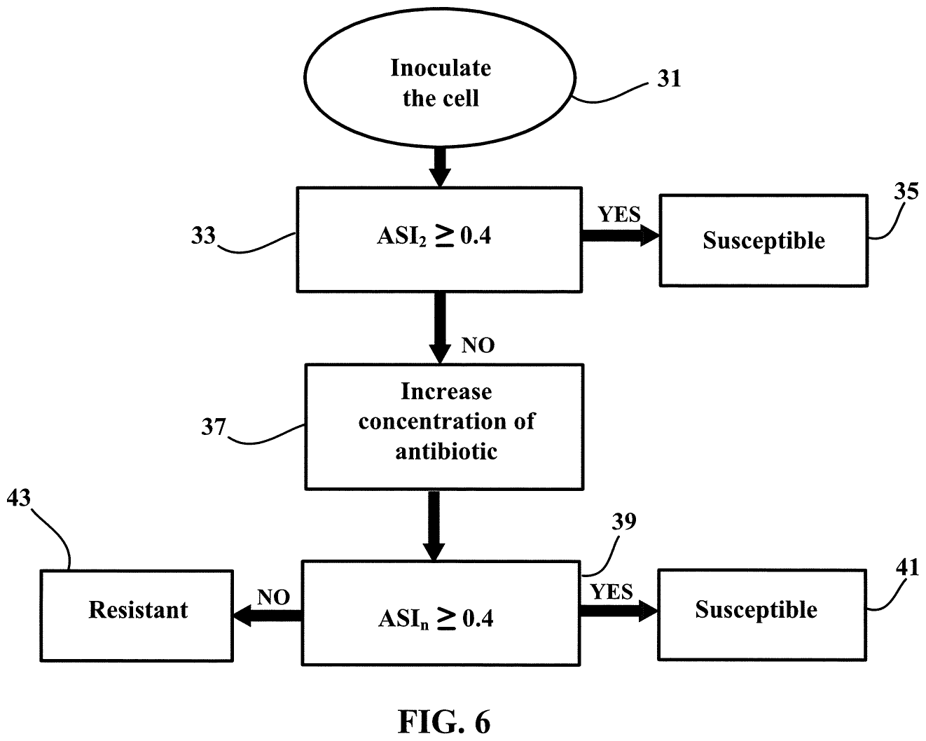

Importantly, such a monitoring also includes a process that along with all the other aspects of the present invention, aids the analysis in a rapid fashion of the induced currents via a resultant antibiotic susceptibility index (ASIₙ), as best detailed in the description for FIG. 6 below.

It is also to be noted that while phenazine methosulfate (PMS) is disclosed as a beneficial mediator used to provide working embodiments herein, it is to be appreciated that other electron transfer mediators can also be utilized where appropriate. For example, mediators, such as, for example, phenazines, flavins, quinones, ferricyanide and ferrocyanides, ferric EDTA, thionine, enzymes using nicotinamide adenine dinucleotide (NAD) and its reduced form NADH, and other electron transfer mediators can also be incorporated without departing from the spirit and scope of the invention.

In addition, pathogens that can be interrogated by a method/system herein include, but are not limited to, Acinetobacter baumannii, Staphylococcus aureus, Escherichia coli, and Klebsiella pneumoniae. Antibiotics utilized with distinct mechanisms of action include an aminoglycoside (tobramycin, protein synthesis), carbapenems (imipenem, cell wall synthesis), a penicillin (oxacillin, cell wall synthesis), and fluoroquinolones (ciprofloxacin, DNA transcription).

Although pathogens and antibiotics are particularly disclosed herein, other strains of known and uncharacterized pathogens and antibiotics with similar or different mechanisms of action can also be incorporated without departing from the spirit and scope of the invention. In addition, beneficial compounds/chemicals used to illustrate the embodiments herein include microbiological growth media, for example, Mueller Hinton (MH), broth, Tryptic soy agar, Sodium Chloride etc.

### Specific Description

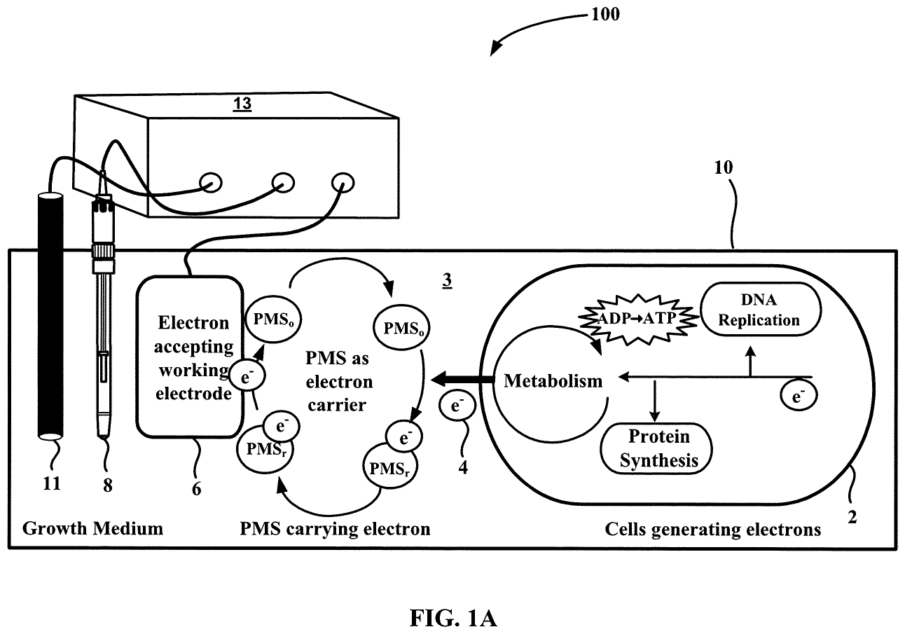

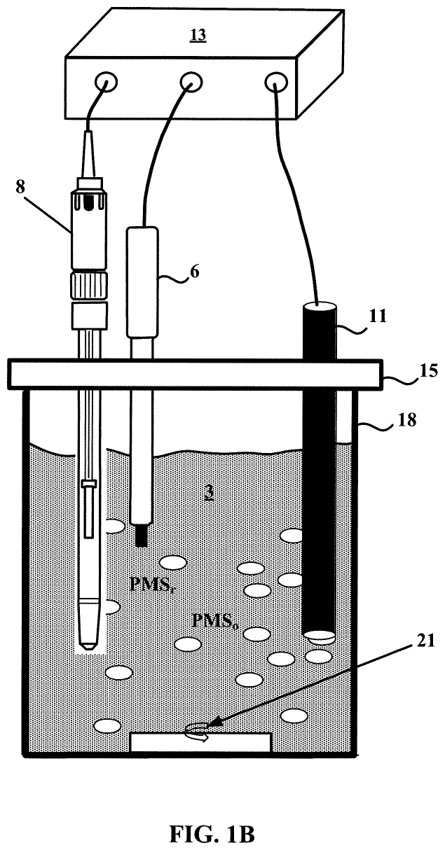

Turning to the drawings, FIG. 1A illustrates a general arrangement of an electrochemical apparatus/system of the invention herein, as generally referenced by the numeral 100. Specifically, the electrochemical apparatus/system 100 includes an operating electrochemical cell 10 structure (well/container), configured to beneficially receive introduced microorganism 2 disposed in a solution 3, desired mediators, such as phenazine methosulfate (denoted as PMS), and two or more electrodes (e.g., Ref Characters 6, 8, 11), wherein such two or more electrodes 6, 8, 11 are often arranged overall as a potentiostat design (generally denoted overall as Ref. Char, 13) for analysis as known to those of ordinary skill in the art. However, other configurations for electrode arrangements can also be utilized where warranted so as to comport with working arrangements of the invention. Moreover, while PMS is a preferred electron transfer mediator (PMSₒ), as discussed above in the general description, it is also to be appreciated that the mediator itself can be any beneficial oxidant that can accept electrons and be reduced (PMSᵣ) by interacting with a microorganism 2 so as to provide current responses when received by a desired electrode.

Regarding operating aspects for the electrochemical apparatus/system 100 configuration of FIG. 1A, such electron transfer mediators (PMSₒ), are beneficially incorporated in a solution 3 (see FIG. 1B) and such electron transfer mediators (PMSₒ) interacts with the respiratory pathway of the microorganism 2 and the reduction of the mediators (PMSᵣ) provides electrons 4 so as to be transported to a working electrode 6 for electrochemical analysis, i.e., a monitored respiration-induced signal (e.g., a current) resulting from the absence and presence of antibiotics configured with distinct mechanisms of action or of differing concentrations. Such signals are indicative of the effectiveness of applied drugs (e.g., antibiotics) to pathogens, i.e., the resistance or susceptibility of certain pathogens to the application of drugs for treatment/scientific purposes. For example, a pathogen (e.g., a bacterium, as denoted as microorganism 2, as shown in FIG. 1A) subjected to, for example, an antibiotic, and is showing resistance to the antibiotic, i.e., no reduction in pathogen vital metabolic processes, results in de minimis change in current responses. Specifically, it results in current responses as compared to control current responses with no substantial differential. In contrast, a pathogen that is subjected to antibiotic that substantially affects vital metabolic processes and/or up to arresting metabolic processes of the pathogen, results in a measured currents substantially and/or dramatically less than current responses and thus is deemed a susceptible microbe to a particular microbe. Importantly, such a monitoring also includes a process that along with all the other aspects of the present invention, aids the analysis in a rapid fashion, such currents via a resultant antibiotic susceptibility index (ASIₙ), as best detailed in the description for FIG. 6 below.

Turning to FIG. 1B in further clarifying the arrangement for the system 100 of FIG. 1A as well as the establishment of more detailed embodiments disclosed infra, electrodes (working electrode 6, reference electrode 8, counter electrode 11) often glassy carbon electrodes were polished using 0.05 μm alumina suspension. Such electrodes were then rinsed with 70% ethanol, DI water and sterilized by UV light for 30 min before insertion into the cell 10. The potential of a reference Ag/AgCl wire was measured in a growth media, such as MH, relative to a saturated Ag/AgCl reference electrode 8 before each experiment. A sterile container (e.g., a plastic cuvette) 18 was utilized and was fitted with a cap (e.g., a custom 3D-printed cap) 15 that was used to position the electrodes 6, 8, 11 in the electrochemical cell 10, as shown in the schematic diagram for the electrochemical arrangement shown in FIG. 1B. At the end of each experiment, the container 18 and the electrodes 6, 8, 11 and cap 15 were sterilized in 70% ethanol for 30 min. The caps and Ag/AgCl wires were rinsed with autoclaved DI water and dried before each use.

With respect to further specific aspects of the experiments disclosed herein, microbiological growth media, for example MH, were supplemented with about 15 μM of an electron transfer mediator, (PMS) and an antibiotic was added to the electrochemical cell 10 (2.5 ml working volume) as was generally illustrated in FIG. 1A and FIG. 1B. For initial quality control, cyclic voltammetry scan was recorded from 0.5 V to −0.5 V then back to 0.5 V using the potentiostat 13 configuration. The potentiostat controls the working electrode voltage relative to the reference electrode.

For antibiotic susceptibility experiments, the working electrode 6 was controlled at 0 V_{Ag/AgCl}. The current can also be measured and analyzed for determining resistance/susceptibility of pathogens to drugs (e.g., antibiotics) using other devices such as a multiplexer or any interface instrument that can receive one or more data inputs and forward it to an output for analysis and feedback. In particular, such one or more data inputs can be directed to a processor, signal processor, computer-based system, etc. and thereafter/correspondingly any such interface instrument can receive instructions from, such processing instruments as better detailed in the description for the embodiment of FIG. 14 below. It is also to be noted that any induced currents undergo a decision methodology that can be interpreted by such a configured processor, signal processor, computer-based system, etc. for aiding in the determination of antibiotic susceptibility/resistance, such as, the methodology better detailed in the description of FIG. 6 below.

Turning back to the discussion for FIG. 1B but also FIG. 1A, an initial baseline was established in the MH media, PMS, and antibiotic mixture for 50-60 min before inoculating the electrochemical cell with a bacteria cell 2. On placing the bacteria cell 2 (again see FIG. 1A) into the electrochemical cell 10, the bacteria cell 2 undergoes metabolic reactions and generates electrons 4 through metabolic processes, as was generally described above. The electron transfer mediator used herein, e.g., PMS, thereafter, operates as an electron carrier in solution 3, and provides generated electrons 4 to the working electrode 6 (herein was aided by stirring mechanism 21, as shown in FIG. 1B). The working electrode 6 thus accepts the electrons provided by the reduced mediators (PMSᵣ) so as to generate currents and returns the mediator back to its oxidized form (PMSᵣ) to keep the monitoring of respiration induced currents if viable.

Tests disclosed in the example embodiments herein for the purpose of illustration were conducted with an inoculum of 0.1 OD₆₀₀. A cell density equating to 0.1 OD₆₀₀ was determined for each species by serial dilution. Briefly, overnight cultures were adjusted to an OD₆₀₀ of 0.1 and then serially diluted (10⁰ to 10⁻¹¹) in a multi-well plate before transferring 10 μL of each dilution to separate TSA plates and grown overnight for 16-18 h. The colony forming units per mL (CFU/mL) were quantified using a drop plate method as known to those skilled in the art. Each measurement was replicated at least four times.

To prepare, for example, an antibiotic such as Ciprofloxacin for testing, such an antibiotic was dissolved in 0.1 N HCl and then diluted in a microbiological growth medium, e.g., MH. The electron transfer mediator, for example, PMS and the antibiotic oxacillin were prepared fresh for each use. Antibiotic tobramycin was stored at 4° C. for <1 week. The antibiotic imipenem stock solutions were prepared and frozen; a fresh stock solution was thawed for each use. Bacteria including Acinetobacter baumannii (strains 35, 78, 83, and 102), Staphylococcus aureus (strains 29213 and 43300), Escherichia coli (strains 61 and 77) and Klebsiella pneumoniae (strains 34 and 115) were utilized in the embodiments herein.

The antibiotic susceptibilities measured through the embodiments disclosed herein for all bacteria strain isolates chosen herein for simplicity, were further verified experimentally using a high throughput microdilution antibiotic susceptibility assays in a microbiological growth medium, such as, MH media following Clinical and Laboratory Standards Institute (CLSI) guidelines (known to those skilled in the art) with 16-18 h incubations at 37° C. Testing for S. aureus susceptibility to oxacillin differed with incubation at 30° C. for 18-24 h in MH media supplemented with 2% NaCl. The minimum inhibitory concentration (MIC) was defined as the lowest concentration of antibiotic for which culture did not grow by visual inspection. Susceptibility and resistance were indicated by established breakpoints (i.e., the concentrations at which bacteria are susceptible to successful treatment with a drug/antibiotic) from CLSI as shown in Table 1 below, wherein antibiotic resistance (AR) and expected resistance is defined for the different bacteria isolates (species).

TABLE 1

| | Total | | |
|---|---|---|---|
| | Number | | |
| | AR | AR Alleles | |
| Species | Genes | Identified | Expected resistance |
| Acinetobacter | 10 | strB, bla_{TEM-1D}, | aminoglycosides, β-lactams, macrolide- |
| baumannii | | aph (3′)-Ic, adc- | lincosamide-streptogramins, |
| | | 25, mph(E), | sulfonamides |
| | | bla_{OXA-66}, bla_{OXA-72}, | |
| | | strA, sul2, | |
| | | msr(E) | |
| Acinetobacter | 9 | adc-25, mph(E), | aminoglycosides, β-lactams, macrolide- |
| baumannii | | bla_{SHV-5}, sul1, | lincosamide-streptogramins, |
| | | bla_{OXA-71}, dfrB1, | sulfonamides, trimethoprim |
| | | aac (3)-IIa, | |
| | | aadA11, msr(E) | |
| Acinetobacter | 15 | bla_{NDM-1}, dfrA1, | aminoglycosides, β-lactams, macrolide- |
| baumannii | | aph (3′)-Ic, | lincosamide-streptogramins, |
| | | mph(E), armA, | phenicols/bicyclomycins, rifampicin, |
| | | arr-3, per-7, | sulfonamides, tetracyclines, trimethoprim |
| | | sul1, bla_{OXA-23}, | |
| | | bla_{OXA-69}, tet(B), | |
| | | strA, sul2, | |
| | | cmlA1, msr(E) | |
| Acinetobacter | 9 | adc-25, armA, | aminoglycosides, β-lactams, macrolide- |
| baumannii | | catB8, mph(E), | lincosamide-streptogramins, |
| | | msr(E), bla_{OXA-66}, | phenicols/bicyclomycins, |
| | | strA, strB, | sulfonamides |
| | | sul1 | |
| Staphylococcus | na | na | na |
| aureus | | | |
| Staphylococcus | na | na | β-lactams (methicillin, oxicillan) |
| aureus | | | |
| Staphylococcus | na | na | β-lactams (methicillin, oxicillan) |
| aureus | | | |
| Escherichia | 9 | aac (3)-IIa, | aminoglycosides, β-lactams, macrolide- |
| coli | | aadA5, bla_{CMY-6}, | lincosamide-streptogramins, |
| | | dfrA17, mph(A), | sulfonamides, tetracyclines, |
| | | bla_{NDM-1}, bla_{OXA-1}, | trimethoprim |
| | | rmtC, sul1, | |
| | | tet(A) | |
| Escherichia | 10 | aac (3)-IId, | aminoglycosides, β-lactams, macrolide- |
| coli | | aadA5, dfrA17, | lincosamide-streptogramins, |
| | | mph(A), strA, | sulfonamides, tetracyclines, |
| | | strB, sul1, sul2, | trimethoprim |
| | | bla_{TEM-52B}, tet(A) | |
| Escherichia | 14 | aac (6′)-Ib, | aminoglycosides, β-lactams, |
| coli | | aadA1, aadA2, | sulfonamides, tetracyclines, |
| | | dfrA12, dfrA14, | trimethoprim |
| | | bla_{KPC-3}, bla_{OXA-9}, | |
| | | strA, strB, | |
| | | sul1, sul2, sul3, | |
| | | bla_{TEM-1A}, tet(A) | |
| Escherichia | 0 | na | na |
| coli | | | |
| Klebsiella | 9 | aac (3)-IId, | aminoglycosides, β-lactams, macrolide- |
| pneumoniae | | catB3, bla_{IMP-4}, | lincosamide-streptogramins, |
| | | mph(A), oqxA, | phenicols/bicyclomycins, quinolones, |
| | | qnrB2, bla_{SHV-11}, | sulfonamides |
| | | sul1, bla_{TEM-1B} | |
| Klebsiella | 13 | aph (3′)-Ia, aph | aminoglycosides, β-lactams, macrolide- |
| pneumoniae | | (4)-Ia, catA1, | lincosamide-streptogramins, |
| | | cmlA1, dfrA12, | phenicols/bicyclomycins, quinolones, |
| | | bla_{KPC-3}, mph(A), | sulfonamides, trimethoprim |
| | | ompK35, oqxA, | |
| | | oqxB, sul1, sul3, | |
| | | bla_{TEM-1A} | |

For performing example experimental tests disclosed in the embodiments herein for further reduction to practice, an overnight culture of the bacteria utilized was prepared. Such a culture was prepared by transferring, for example, two colonies from an agar streak plate into 8-ml MH broth and cultured overnight in an air incubator (37° C.) rotating at 120 rpm for 16-18 h. A 5-ml aliquot of culture was adjusted to 0.5 OD₆₀₀ with fresh MH media and then centrifuged at 6000 rpm (4185×g) for 8 min and the supernatant decanted. The pellet was then resuspended in 5-mL of 30° C. (S. aureus experiments) or 37° C. (all others) MH media. After resuspension, 2 mL of the culture was used to verify optical density. Finally, 500 μL of the culture was inoculated to the electrochemical cuvettes by pipette through the cap inoculation port (2.5-ml final volume in the cuvette).

Growth curves of the bacteria were recorded under such initial conditions to the electrochemical experiments (MH media supplemented with 15 μM PMS and antibiotic). The wells were inoculated to 0.1 OD₆₀₀ for a total volume of 200 μL. The plates were continuously mixed by an orbital motion and maintained at 37° C. or 30° C. for S. aureus and oxacillin. Absorbance growth curves were recorded at 600 nm for 24 h and growth curves were presented as the average of three technical replicates with standard deviation.

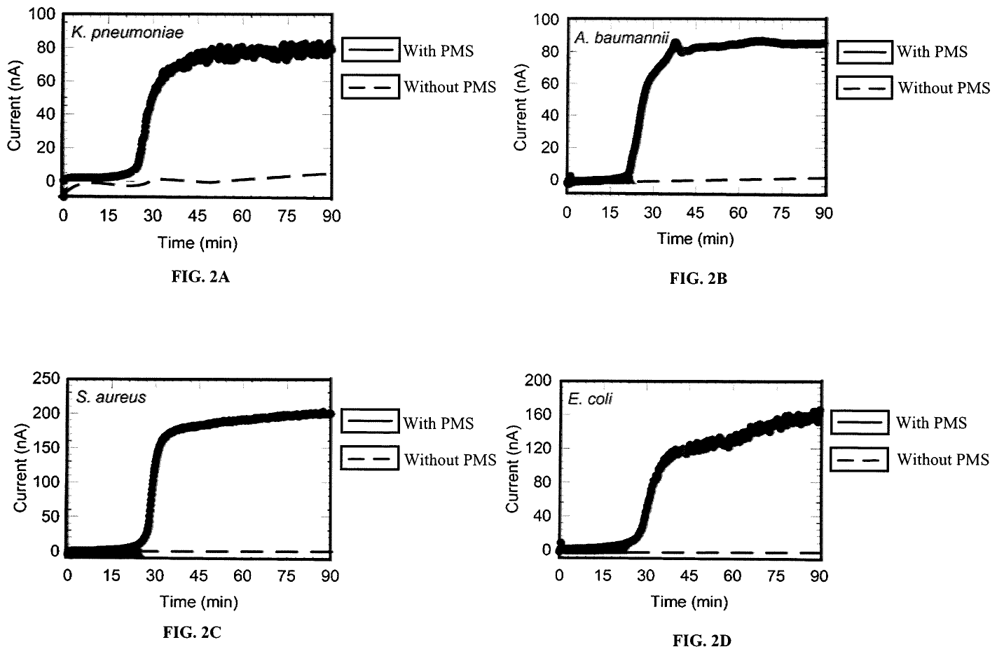

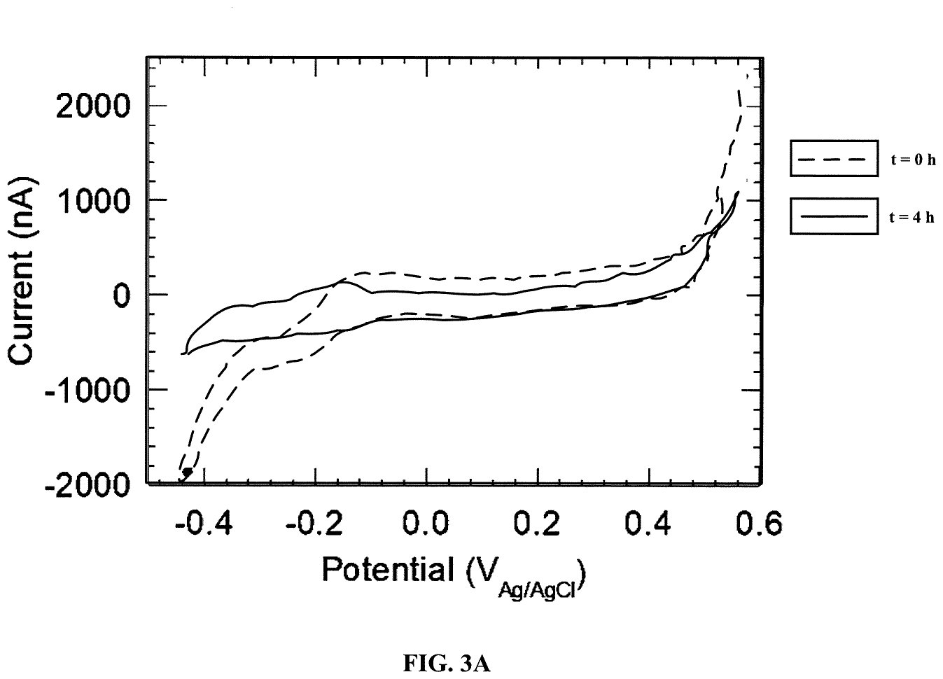

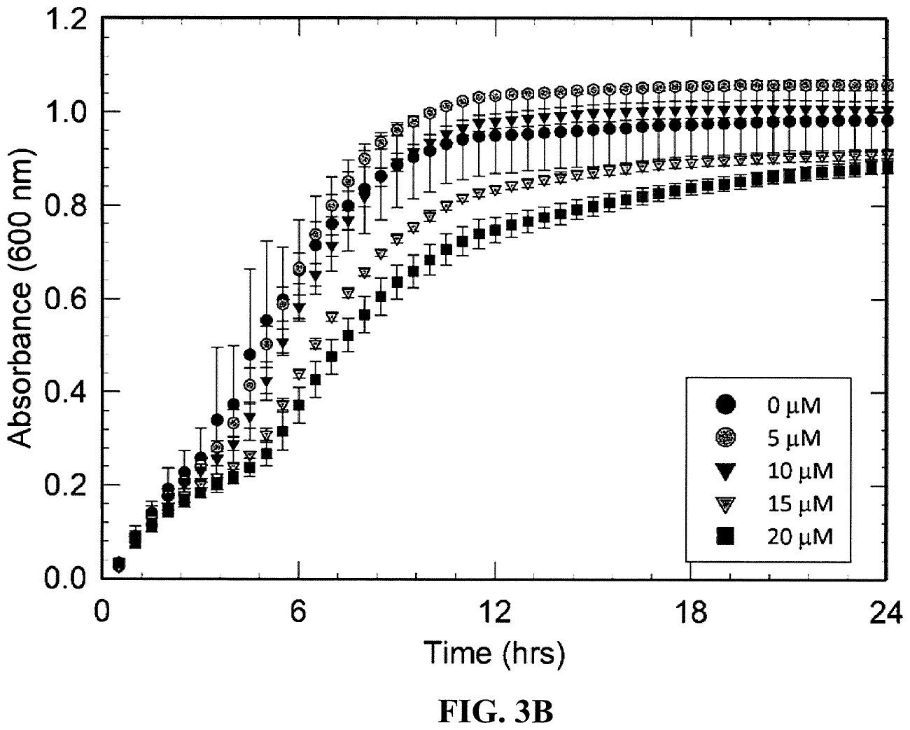

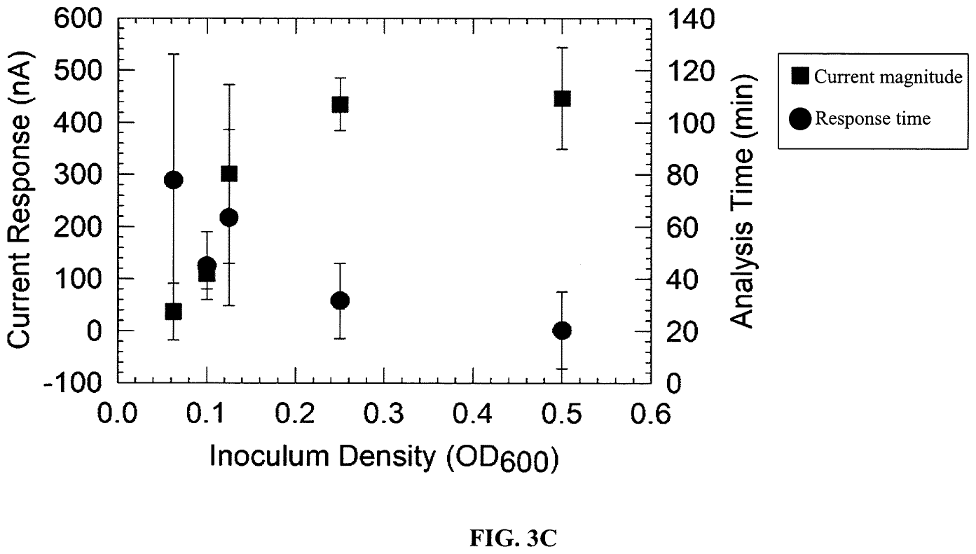

For initial experiments, mediator-free background current measurements were collected for each strain. For all the tested strains, with results as shown in Table 2, there was no detectable current without the presence of PMS and thus no appreciable background current and most if not all measured current is due to the microbial reduction of PMS and its oxidation at the inert electrode surface, as shown in FIG. 2A for bacterial strain of K. pneumoniae, in FIG. 2B for bacterial strain of A. baumannii, in FIG. 2C for bacterial strain of S. aureus, and in FIG. 2D for bacterial strain of E. coli. Similar conclusions can be drawn from cyclic voltammetry performed on the electrochemical system before inoculating and at the end of the experiment to show that PMS was the sole electron mediator and that the only electrochemical reaction that occurs during the measurement is the reduction and oxidation of PMS as shown by FIG. 3A. However, while strains tested herein included a mediator, such as, PMS, it is to be appreciated that the invention herein can also be utilized without an exogenous mediator. For example, the invention herein can also incorporate a bacterium, e.g., P. aeruginosa species, that endogenously produce mediators called phenazines that facilitate extracellular electron transport capabilities. In addition, in cases of limited electron transfer rates from such species, the interrogation of such species can be augmented with exogenous mediators such as PMS to enhance signals where warranted.

In FIG. 3A, voltammogram of 15 μM PMS, initially fully oxidized, in MH media by a glassy carbon electrode, with a graphite counter and Ag/AgCl reference at 10 mV/s from 0.57 to −0.43 V_{Ag/AgCl} is shown. Cyclic voltammetry was recorded before inoculating with E. coli (t=0 h) and after the experiment (t=4 h). The second cycle of the cyclic voltammetry is shown in FIG. 3A with observed PMS reduction and oxidation peaks centered around −0.13 V_{Ag/AgCl}. The reduction and oxidation peaks of PMS do not shift during the experiment providing evidence that PMS is not chemically altered during the microbial reduction. Additionally, it is the sole electron mediator throughout the experiment, since the only peaks present in the media are from PMS, and that the only reaction occurring inside of the system is the oxidation and reduction of PMS given that this is a reversable reaction on the electrode surface.

In the embodiments herein, the electrodes detect the reduction of the electron transfer mediator, which is then recorded as current which means that the electrical current measurements respond to the electron transfer reduction. The minimum change in the electron transfer mediator, for example, the phenazine methosulfate (PMS) concentration that corresponds to measurable change in current in the READAS assay is as per the calculations below.

Consider a measured current increased above 6× the standard deviation of the background current which was passing above 1 nA in 14.3 minutes. This current magnitude of I nA and response time of 14.3 minutes is used to estimate the minimum change in PMS concentration. First current is converted from nanoamperes to amperes.

$$1 nA \times \frac{1 A}{{10}^{9} nA} = {10}^{- 9} A$$

Using Faraday's constant (96485 C/mol), the current (A=C/s) is converted to moles of electrons per second.

$$\frac{1 \times {10}^{- 9} C}{s} \times \frac{mol {e}^{-}}{96485 C} = 1.036 \times {10}^{- 14} \frac{mol {e}^{-}}{s}$$

The electron moles are converted to moles of PMS oxidized per second. Reoxidation of PMS reduced by the cells occurs via a 2-electron transfer reaction.

$$\frac{1.036 \times {10}^{- 14} mol {e}^{-}}{s} \times \frac{mol PMS}{2 mol {e}^{-}} = 5.18 \times {10}^{- 15} \frac{mol PMS}{s}$$

Finally, the moles of PMS are divided by the total volume of the reactor (2.5 mL)

$$5.18 \times {10}^{- 15} \frac{mol PMS}{s} \times \frac{1}{0.0025 L} = 2.07 \times {10}^{- 12} \frac{mol}{s L} = 2.07 \frac{pM}{s}$$

Using READAS, current changes above the background level corresponds to the ability to detect changes in PMS concentrations as low as 2.07 pM per second; i.e., a 10 nA current change over 100 minutes corresponds to 207 pM change in PMS concentration.

Values listed in Table 2 below include the estimated minimum inhibitory concentration from this study (μg/mL), the determination of(S) sensitive or (R) resistant microbes according to CLSI criteria, and the CLSI guidelines (μg/mL) for the species being tested. Empty cells were not evaluated.

TABLE 2

| Species | Strain | Tobramycin | Imipenem | Oxacillin | Ciprofloxacin |
|---|---|---|---|---|---|
| Acinetobacter | 35 | 0.5 (S) 16 | | | |
| baumannii | 83 | 128 (R) 16 | | | |
| 5.8 × 10⁷ ± 4.3 × 10⁷ | 78 | | 32 (R) 16 | | |
| | 102 | | 1 (S) 16 | | |
| Staphylococcus | 29213 | 1 (S) 16 | | 0.5 (S) 6 | |
| aureus | 43300 | 128 (R) 16 | | 32 (R) 6 | |
| 1.1 × 10⁸ ± 2.0 × 10⁷ | | | | | |
| Escherichia coli | 61 | 16 (R) 16 | 32 (R) 4 | | |
| 2.7 × 10⁸ ± 1.5 × 10⁷ | 77 | 0.5 (S) 16 | 0.5 (S) 4 | | |
| Klebsiella | 34 | | 2 (S) 4 | | 0.25 (S) 1 |
| pneumoniae | 115 | | 64 (R) 4 | | 64 (R) 1 |
| 2.7 × 10⁸ ± 1.7 × 10⁷ | | | | | |

An example PMS concentration of 15 μM was selected because higher concentrations produced variable degrees of inhibition between strains, as represented by data in FIG. 3B. Error bars represent standard deviation of three replicates. With this PMS concentration of 15 μM, Gram-negative species of bacteria with OD₆₀₀=0.1 (approximately 10⁷ and 10⁸ cells per mL; Table 2) consistently had analysis times in less than 90 min, as illustrated by FIG. 3C. Current response of A. baumannii inoculated at inoculum densities (OD₆₀₀ 0.0625 to 0.5) with 15 μM PMS is shown in FIG. 3C. The current magnitude is shown to increase with increasing cell density while the response time is seen to be decreased with increasing cell density. Error bars are the standard deviation of four replicates in FIG. 3C. Increasing the cell density also contributed to the “inoculum effect” of antibiotic efficacy resulting in the need for higher concentrations of antibiotics to differentiate sensitive and resistant strains.

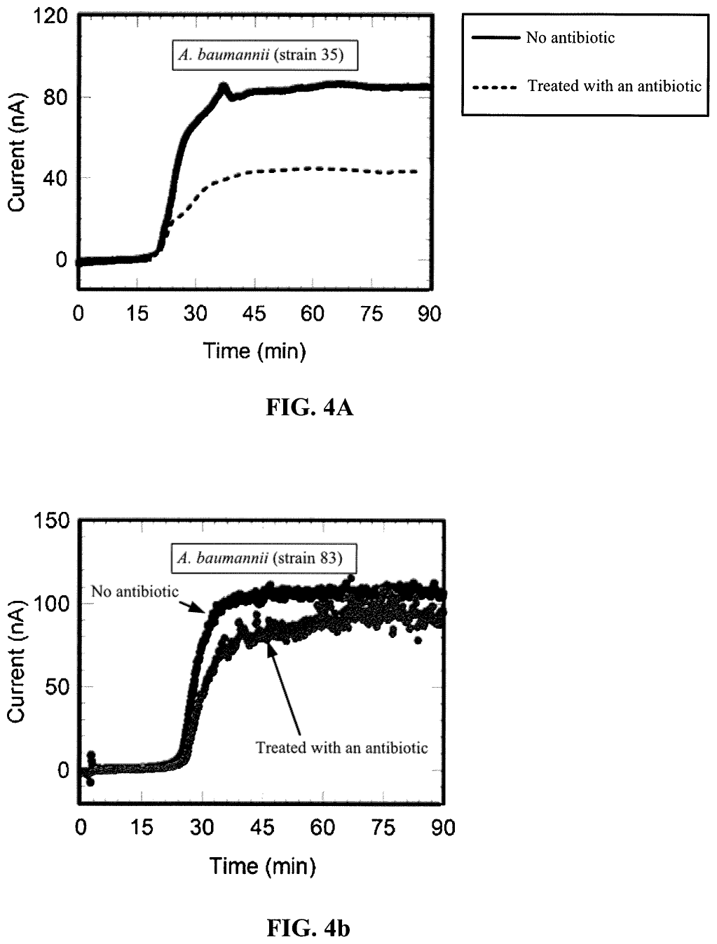

FIG. 4A shows an electrochemical current response of a susceptible A. baumannii strain while FIG. 4B shows an electrochemical current response of a resistant A. baumannii strain. It is to be noted that a CLSI resistance breakpoint concentration is a defined concentration of a susceptible antibiotic that treats the bacteria. The bacteria strains of A. baumannii were tested with the antibiotic tobramycin using a 2×CLSI resistance breakpoint concentration which is 32 μg/mL. The current response was characterized by a typical sigmoidal curve with a delay of approximately 20-30 minutes before detecting current increase above background level. Current response for each strain was compared in the presence of an antibiotic known as treatment (experiment) and in the absence of antibiotic challenge known as control (experiment). The susceptible strain treated with the antibiotic exhibited a 50% decrease in current compared to the control as represented in FIG. 4A. In contrast, as represented in FIG. 4B, the resistant strain showed a similar current magnitude profile over time in the absence of tobramycin challenge.

The current responses measured follows a sigmoidal curve structure. The magnitude of the current response was defined as the current magnitude at the inflection point between the exponential growth and the saturated current response. In the embodiments herein, an antibiotic susceptibility index (ASIₙ) is disclosed as a unitless index that is used to compare the reduction of measured current when a strain is exposed to varying antibiotic concentrations compared to control experiments. In the example embodiments herein, ASIₙ is used in the decision methodology to determine if a given bacteria strain (known or uncharacterized) is susceptible to or resistant to either a range of different antibiotics or different concentrations of a given antibiotic.

For example, in FIG. 4A, the susceptible strain of A. baumannii (strain 35) had an ASI₂ of 0.5 while the resistant strain (strain 83) shown in FIG. 4B had an ASI₂ of 0.3. The ASI₂ analysis times for the strains were 35 and 33 min respectively, as determined from about the time at the inflection point of the exponential growth and saturated current responses.

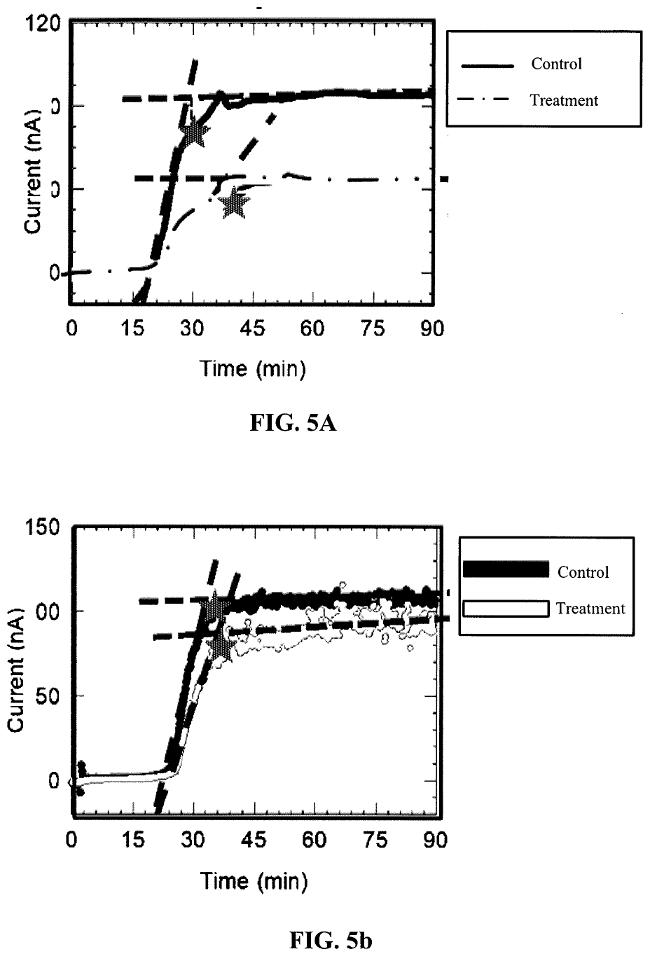

FIG. 5A and FIG. 5B illustrates example raw current responses after inoculating the electrochemical cell with 0.1±0.01 OD₆₀₀ A. baumannii susceptible (FIG. 5A) and resistant strains (FIG. 5B). In FIG. 5A and FIG. 5B, best fit lines to the exponential and stationary growth phase are shown by vertical dashed lines, matching the intersection of the best fit lines to the current indicated by horizontal dashed lines and the intersection point of the best fit lines are indicated by a star icon (used to record the current response (y-axis) of the respective control and treated cultures). The ASIₙ analysis time is the time (x-axis) of the intersecting lines. The difference in the raw current response is presented as the ASIₙ. To calculate the ASIₙ, first, best fit lines (in FIGS. 5A and 5B) are fitted to the exponential phase and stationary phases of both the parallel control and treated current responses. Starting with the control, the intersection point is followed directly down until crossing the raw data. The point at which the current matches the intersection point is the current magnitude (in nA) and analysis time (in min) of the control current response. Following the same procedure for the treatment, best fit lines are fitted to the exponential and stationary phases of the current response (thick black dashed lines). Next, the intersection point is followed vertically down until crossing the raw current response (light black dashed line). The point in which the blue dashed line crosses the raw current response is the current magnitude and analysis time of the treatment. The ASIₙ is calculated by taking the difference between the control and treatment current magnitude of the intersection point. The analysis time is the time of the intersection point for the resistant and susceptible treatments.

ASIₙ was calculated as shown in the equation below,

$${ASI}_{n} = \frac{{i}_{control} - {i}_{treatment}}{{i}_{control}} \qquad ( 1 )$$

where n refers to the multiple of the antibiotic breakpoint concentration tested, iₜᵣₑₐₜₘₑₙₜ is the current of the strain under antibiotic exposure, i_{control} is the current from the same strain in the untreated control. Based on results from 16 species-antibiotic combinations, an ASIₙ of 0.4 or greater was selected empirically as a suitable threshold for distinguishing between antibiotic-susceptible (≥0.4) and -resistant (<0.4) strains. However, while an ASIₙ of 0.4 or greater was deemed as a suitable threshold for distinguishing between antibiotic-susceptible (≥0.4) and -resistant (<0.4) strains, it is also important to note that new microorganisms/samples from new environments/potential interference from media components, etc. can modify this empirical index/threshold as needed.

The decision methodology for determining antibiotic susceptibility and resistance is as shown in FIG. 6. After the initial steps of filling the electrochemical reactors with, for example, a growth medium and the strain, i_{control} is observed without any treatment to the strain. Then the antibiotic with a concentration of 2 times the antibiotic breakpoint concentration is introduced in the electrochemical reactor to inoculate 31 the strain. The electric current response is continuously observed, for example, using a potentiostat configuration, as shown above in describing FIG. 1A and FIG. 1B, to get the value of iₜᵣₑₐₜₘₑₙₜ. ASI₂ 33 is then calculated using the equation above (1). If the calculated value of ASI₂ is greater than or equal to 0.4 then the strain is determined to be susceptible 35 to the concentration of the antibiotic selected for the experiment. If the value of the is less than 0.4, then the concentration of the antibiotic is increased 37, and the experiment then includes and entails: recalculating the ASIₙ 39 and if the calculated value of ASIₙ is greater than or equal to 0.4 then the strain is determined to be susceptible 41 to the concentration of the antibiotic selected for the experiment. If the value of the ASIₙ is less than 0.4, then the strain is determined to be resistant 43 to the concentration of the antibiotic selected for the experiment. This analysis could also loop a number of times if necessary.

As to be described in detail for the system shown in FIG. 14 below, such a methodology described for FIG. 6 can be equally utilized in a novel fashion to accommodate analysis of known or uncharacterized bacteria stains in the manner described above with respect to FIG. 6 for a multiple-reactor (well) system arrangement. For example, using the methodology of FIG. 6, in such a multi-well reactor (well) arrangement, a number of reactors/wells can be configured with, for example, control solutions for testing, a same or different pathogen, with selected wells being treated with same or differing antibiotics and/or same or differing concentrations of such antibiotics for rapid analysis with increased certainty of the susceptibility and/or resistance of such strains to antibiotics. To aid in such an application, such a multi-well (reactor) system be coupled with instrumentation such as processor 201, as shown in FIG. 14 to expedite/aid pathogen well selection and antibiotic application/concentrations. As an additional arrangement and using same coupled instrumentation, for a multiple reactor (well) system, particular reactors can also be arranged with differing pathogens and with desired reactors being treated with a same or a different antibiotic and/or same or differing concentrations of such antibiotics for a variation of the rapid analysis methodology disclosed herein.

Turning back to FIG. 6 and to reiterate for emphasis, the experiment is repeated either until the strain is susceptible to a particular concentration of the antibiotic or until 24 times the breakpoint antibiotic concentration is used for the experiment. If ASI₂₄ is also not greater than or equal to 0.4, then the strain is determined to be resistant to the antibiotic.

The READAS response time (t_{6σ}) was defined as the time at which the current response increases above the average background current (M) by six multiples of standard deviation (i.e., M+6σ). The time to determine ASIₙ (analysis time) was defined as the time at the inflection point of the exponential growth and saturated current responses and denoted as ASIₙ Analysis Time. A two-sided Wilcoxon rank-sum test was used to determine if the ASIₙ and analysis time was statistically different between susceptible and resistant strains. Data were represented as means and standard errors of at least four biological replicates.

Tests were conducted for which the species or resistance phenotype was not known beyond the fact that one set was a Gram-positive and the other Gram-negative, and the antibiotics to be tested were oxacillin and imipenem, respectively. The isolates were provided as coded streak plates. Because the species were unknown, the breakpoint concentration of oxacillin and imipenem were estimated from the known breakpoints of the four previously tested strains (as shown in Table 1; 6 μg/mL for oxacillin, S. aureus and 6 μg/mL imipenem for Gram-negative strains). Following the decision methodology for determining antibiotic susceptibility and resistance of READAS, the strains were first tested with 2× the resistance breakpoint concentration (12 μg/mL oxacillin or imipenem) and the ASI₂ was measured. This was increased up to 24× the resistance breakpoint concentration. If ASI₂≥0.4 then the strain was considered susceptible. Because it was known that one strain of each species was susceptible while the other resistant, no further testing was done if one of the strains had an ASI₂≥0.4. If neither strain achieved an ASI₂≥0.4, 24× the resistance breakpoint concentration was tested and the ASI₂₄ was measured. The susceptible strain was defined as the strain with an ASI_{24≥0.4}, while the resistant strain had an ASI_{24<0.4.}

The present invention will be more fully understood by reference to the following examples, which are intended to be illustrative of the present invention, but not limiting thereof.

### Examples

### Examples of Antibiotic-Susceptible and -Resistant Strains

### Example 1: Assessing Antibiotic-Susceptible and -Resistant Strains of A. baumannii, S. Aureus, E. coli, and K. pneumoniae

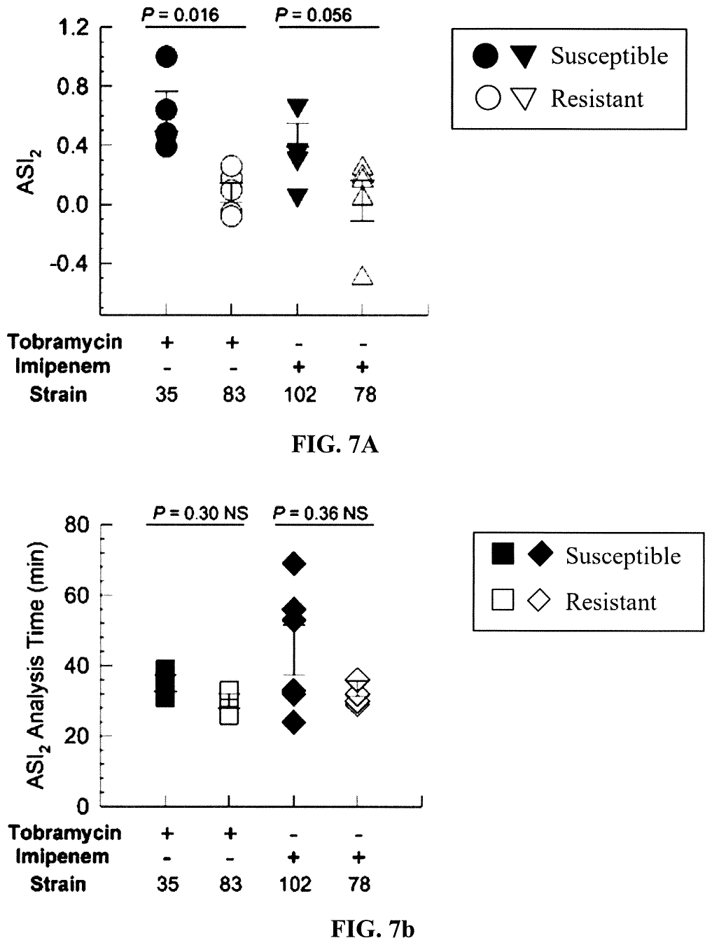

As an example method of operation, antibiotic-susceptible and -resistant strains of A. baumannii selected from Table 2 were assessed in the presence of 2×CLSI resistance breakpoint concentrations of either the bacteriostatic antibiotic tobramycin or the bactericidal antibiotic imipenem. The results of this example operation are as shown in FIG. 7A and FIG. 7B, wherein error bars represent standard error. The tobramycin susceptible strain (35) exhibited an average ASI₂ of 0.63±0.13 (average±SE) whereas the tobramycin-resistant strain (83) had an ASI₂ of 0.08±0.06 as shown in FIG. 7A. For imipenem treatment, the susceptible strain (102) had an ASI₂ of 0.47±0.08, whereas the average ASI₂ of the resistant strain (78) was mostly unaffected (0.02±0.13) compared to no-antibiotic control. In both cases, no statistically significant difference (P=0.30 and P=0.36, respectively) was detected in the analysis time for these assays as supported by results in FIG. 7B. ASI₂ analysis time ranged from 26-39 min for tobramycin and 24-69 min for imipenem. These results are included in Table 3 below.

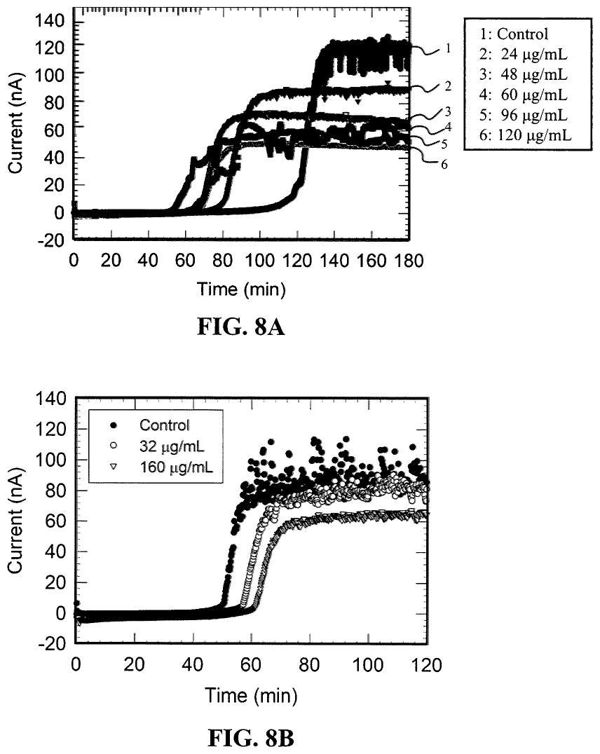

As a second example method of operation, the S. aureus strains selected from Table 2 were assessed. Initially, a 2×CLSI resistance breakpoint concentrations for two antibiotics oxacillin (12 μg/mL) and tobramycin (32 μg/mL) were used for the example method of operation. The ASI₂<0.4 and it was concluded that the concentration of the antibiotics was insufficient to classify the strains as antibiotic-susceptible or -resistant. S. aureus was then treated with 4×, 6×, 8×, 10×, and 20× the breakpoint concentration of oxacillin and was compared to the control as depicted in FIG. 8A. The current response decreased with increasing oxacillin concentration except at low doses where no effect or an increase in the current is observed. S. aureus was also treated with 4× and 10× the breakpoint concentration of tobramycin and was compared to the control depicted in FIG. 8B. Increasing the tobramycin concentration decreased the current magnitude to ASIₙ≥0.4 and increased the response time. The efficacy of antibiotic was proportional to the concentration when a high cell density (1.1×10⁸ CFU/mL) was used.

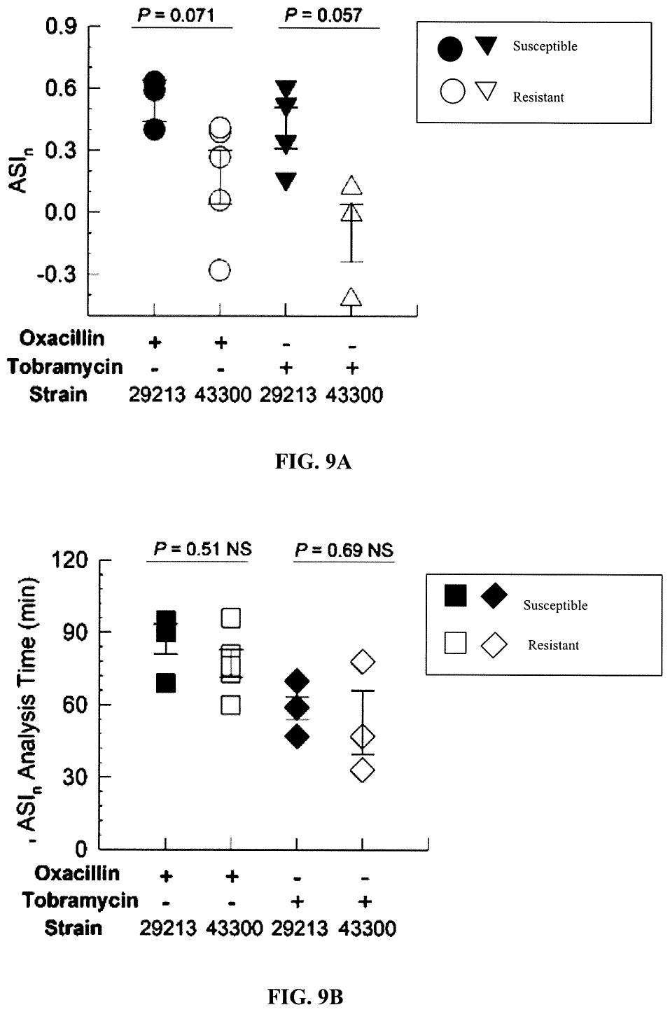

Consequently, as the concentrations were increased to 20×(120 μg/mL) for oxacillin and 10×(160 μg/mL) for tobramycin as shown in FIG. 9A and FIG. 9B (Error bars represent standard error), it resulted in an average ASIₙ≥0.4. The tobramycin susceptible strain (29213) exhibited an average ASI₁₀ of 0.41±0.2 whereas the resistant strain (43300) showed a higher current under antibiotic challenge (ASI₁₀ of −0.10±0.14) as shown in FIG. 9A. For oxacillin treatment, the susceptible strain (29213) exhibited an average ASI₂₀ 0.54±0.07 whereas the resistant strain (43300) exhibited an average ASI₂₀ 0.17±0.13 as shown in FIG. 9B. The ASIₙ analysis time for classifying the strains as resistant or susceptible for tobramycin treatment was 33-78 min which was 30 min less than needed for detecting oxacillin susceptibility which required 60-96 min. These results are included in Table 3 below.

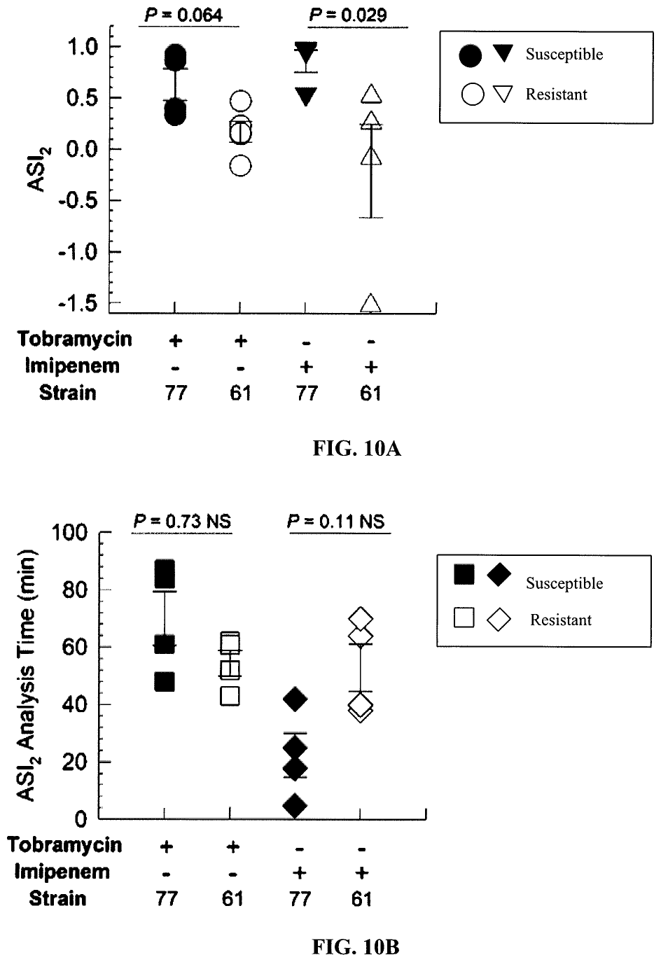

As a third example method of operation, E. coli strains selected from Table 2 were used with 2×CLSI resistance breakpoint concentrations of antibiotics imipenem (8 μg/mL) and tobramycin (32 μg/mL) were used. The tobramycin susceptible strain (77) exhibited an average ASI₂ of 0.62±0.15 compared to an ASI₂ of 0.17±0.1 in the resistant strain (61) as shown in FIG. 10A. The imipenem susceptible strain (77) had an ASI₂ of 0.86±0.11 but the resistant strain (61) had a higher current response under antibiotic challenge (ASI₂ of −0.20±0.45) as depicted in FIG. 10B. In FIG. 10A and FIG. 10B error bars represent standard error. The analysis time under tobramycin treatment ranged from 43-105 min while imipenem ASI₂ analysis time was between 5-40 min for the susceptible and 38-70 min for the resistant as shown in Table 3.

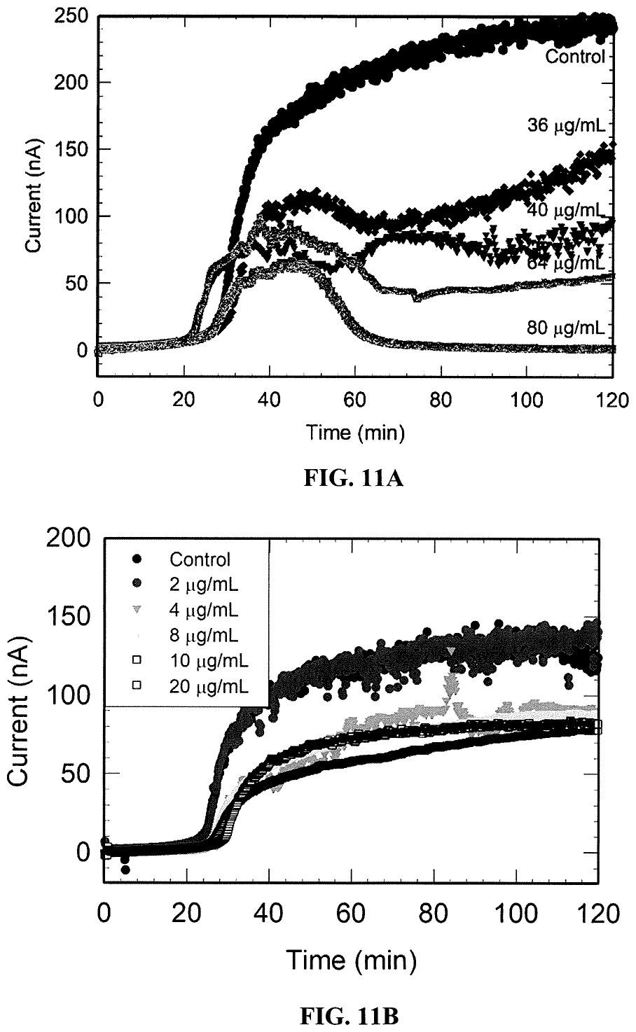

As a fourth example method of operation, K. pneumoniae strains selected from Table 2 were used with 2×CLSI resistance breakpoint concentration. This concentration of the antibiotic was insufficient to classify resistant and susceptible strains for ciprofloxacin and imipenem. K. pneumoniae control was compared to the strain treated with 6×(36 μg/mL), 10×(40 μg/mL), 16× (64 μg/mL), and 20×(80 μg/mL) the breakpoint concentration of imipenem. Increasing the imipenem concentration decreased the current response as shown in FIG. 11A. Consequently, when the concentration was increased to 20×CLSI resistance breakpoint concentration, or 20 μg/mL and 80 μg/mL, respectively, the current returned to background providing a clear differentiation between the susceptible and resistant species in addition to determining the ASIₙ. Using 20× the breakpoint concentration resulted in ASIₙ≥0.4. K. pneumoniae control was also compared to the strain treated with 2×(2 μg/mL), 4×(4 μg/mL), 8×(8 μg/mL), 10×(10 μg/mL), and 20×(20 μg/mL) the breakpoint concentration of ciprofloxacin. Increasing the antibiotic concentration decreased the current magnitude of the susceptible strain to ASIₙ≥0.4 as shown in FIG. 11B. The efficacy of antibiotic proportional to the concentration when a high cell density (2.7×10⁸ CFU/mL) was used.

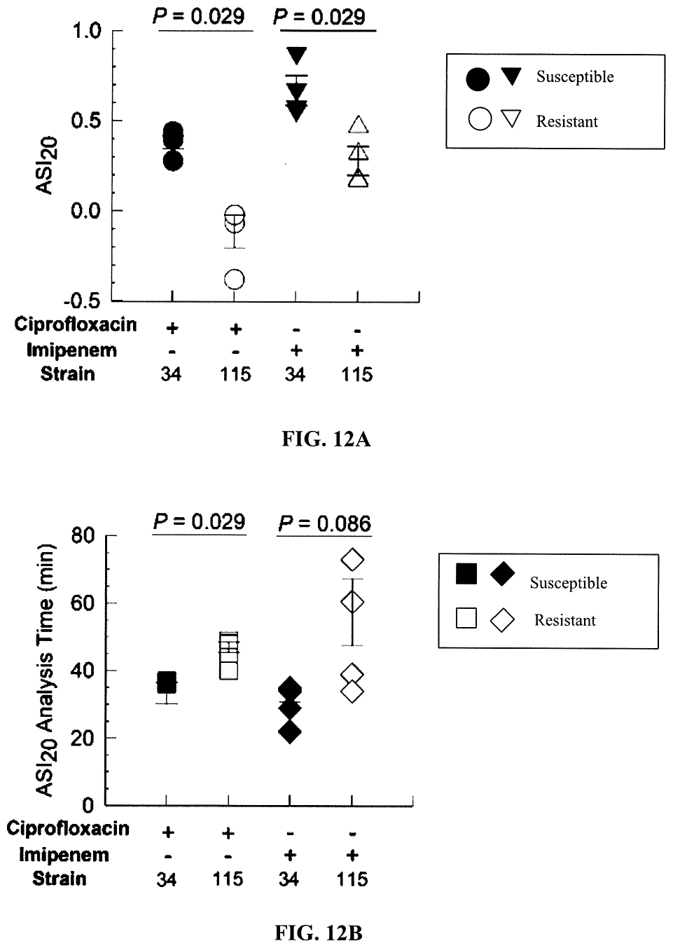

The ciprofloxacin-susceptible strain (34) had an ASI₂₀ of 0.41±0.1, whereas the ciprofloxacin-resistant strain (115) generated a higher current relative to the untreated control (ASI₂₀ of −0.16±0.11) as shown in FIG. 12A. The imipenem-susceptible strain (34) had an ASI₂₀ of 0.67±0.08 compared to an ASI₂₀ of 0.28±0.08 in the resistant strain (115) as shown in FIG. 12B. In FIG. 12A and FIG. 12B error bars represent standard error. For both antibiotics, the analysis time of the susceptible strain was less the resistant strain which was about 22-35 min for imipenem and about 13-37 min for ciprofloxacin, but for both cases, antibiotic susceptibilities were observed in less than 90 min, 14-49 min exactly as shown in Table 3.

TABLE 3

| | | ASIₙ | | ASIₙ | | ASIₙ | | ASIₙ | |
|---|---|---|---|---|---|---|---|---|---|
| | | Tobramycin | t_{6σ} | Imipenem | t_{6σ} | Oxacillin | t_{6σ} | Ciprofloxacin | t_{6σ} |
| Species | Strain | (analysis time) | (min) | (analysis time) | (min) | (analysis time) | (min) | (analysis time) | (min) |
| Acinetobacter | 35 | 0.48 | 3-14 | | | | | | |
| baumannii | | 1 | | | | | | | |
| | | 0.4 | | | | | | | |
| | | 0.64 | | | | | | | |
| | | (35-39 min) | | | | | | | |
| | 83 | −0.06 | 11-22 | | | | | | |
| | | −0.08 | | | | | | | |
| | | 0.17 | | | | | | | |
| | | 0.09 | | | | | | | |
| | | 0.26 | | | | | | | |
| | | (26-33 min) | | | | | | | |
| | 78 | | | 0.04 | 5-24 | | | | |
| | | | | −0.5 | | | | | |
| | | | | 0.24 | | | | | |
| | | | | 0.19 | | | | | |
| | | | | 0.16 | | | | | |
| | | | | (29-41 min) | | | | | |
| | 102 | | | 0.37 | 1-8 | | | | |
| | | | | 0.36 | | | | | |
| | | | | 0.67 | | | | | |
| | | | | 0.31 | | | | | |
| | | | | 0.65 | | | | | |
| | | | | (24-69 min) | | | | | |
| Staphylococcus | 29213 | 0.52 | 12-21 | | | 0.4 | 10-45 | | |
| aureus | | 0.6 | | | | 0.63 | | | |
| | | 0.16 | | | | 0.59 | | | |
| | | 0.34 | | | | (69-95 min) | | | |
| | | (47-70 min) | | | | | | | |
| | 43300 | 0.12 | 7-37 | | | 0.4 | 10-45 | | |
| | | −0.01 | | | | 0.63 | | | |
| | | −0.42 | | | | 0.59 | | | |
| | | (33-78 min) | | | | (69-95 min) | | | |
| Escherichia | 61 | 0.23 | 1-4 | 0.26 | 2-25 | | | | |
| coli | | 0.15 | | −1.52 | | | | | |
| | | 0.47 | | 0.52 | | | | | |
| | | 0.16 | | −0.08 | | | | | |
| | | −0.16 | | (38-70 min) | | | | | |
| | | (43-105 min) | | | | | | | |
| | 77 | 0.92 | 2-22 | 0.98 | 2-22 | | | | |
| | | 0.4 | | 0.97 | | | | | |
| | | 0.86 | | 0.96 | | | | | |
| | | 0.33 | | 0.54 | | | | | |
| | | (48-87 min) | | (5-42 min) | | | | | |
| Klebsiella | 34 | | | 0.56 | 0.2-0.5 | | | 0.39 | 0.5-1 |
| pneumoniae | | | | 0.88 | | | | 0.44 | |
| | | | | 0.67 | | | | 0.41 | |
| | | | | 0.58 | | | | 0.27 | |
| | | | | (22-35 min) | | | | (13-37 min) | |
| | 115 | | | 0.32 | 0.5-1 | | | −0.38 | 0.2-1 |
| | | | | 0.47 | | | | −0.07 | |
| | | | | 0.18 | | | | −0.03 | |
| | | | | 0.17 | | | | 0.02 | |
| | | | | (34-73 min) | | | | (14-49 min) | |

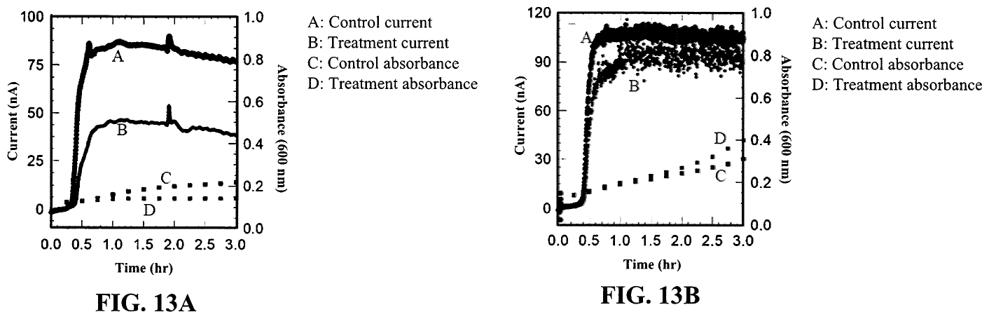

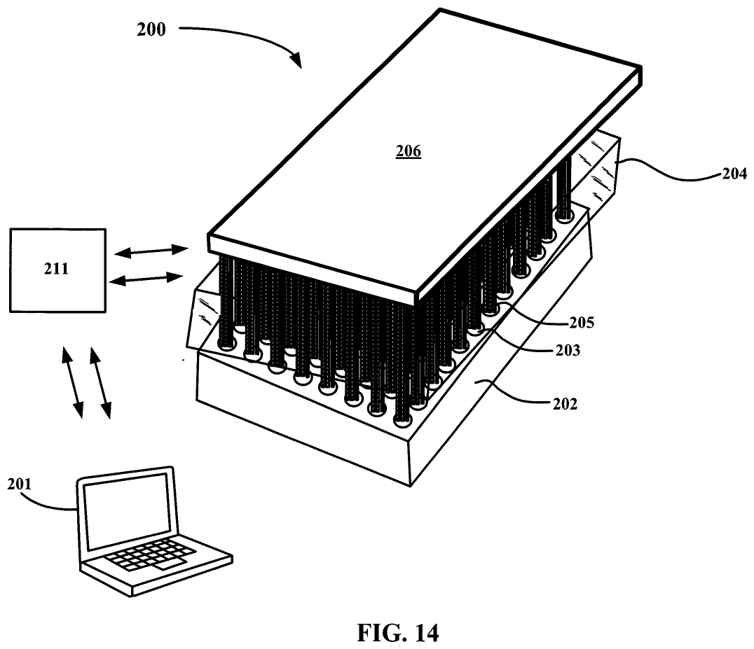

The main advantage of electrochemical antibiotic susceptibility testing disclosed in the embodiments herein is that it allows a more direct measurement of respiration in comparison to traditional reliance on growth of bacterial cultures under antibiotic challenge. To illustrate this difference, parallel electrochemical and absorbance assays for strain A. baumannii (strains 35 and 83) in the presence or absence of tobramycin were performed, results of which are as shown in FIG. 13A and FIG. 13B. For both the susceptible strain in FIG. 13A and resistant strain in FIG. 13B, robust electrochemical responses were detected within 30 min and with large dynamic ranges. In contrast, optical density measurements were relatively limited with clear distinctions not evident until between one to two hours, and overall dynamic ranges were limited during this period.

As for any assay that generates a continuous response variable over time, it is necessary to identify a threshold that distinguishes between antibiotic-susceptible and -resistant strains. Given the variation in analysis times relative to strain, antibiotic, and susceptibility status (as in FIG. 9A, FIG. 9B, FIG. 12A and FIG. 12B), this parameter was not considered as a robust measure upon which to base a threshold. Instead, it is to be noted that the ASIₙ results (as in FIG. 7A, FIG. 7B, FIG. 10A, and FIG. 10B) suggest that robust classification of a susceptible strain can be achieved with a threshold of an ASIₙ≥0.4 while a resistant strain can be classified with a threshold of ASIₙ<0.4 with increasing antibiotic concentration.

### Example 2: Assessing Antibiotic-Susceptible and -Resistant Strains of Gram-Positive and -Negative Bacteria Through Blinded Tests

As another example method of operation, operator blinded tests were conducted using susceptible and resistant strains of Gram-positive and -negative bacteria. Because the species identification was unknown, 6 g/mL oxacillin was chosen as the estimated resistance breakpoint for the Gram-positive strain and 6 μg/mL imipenem was initially chosen as the resistance breakpoint for the Gram-negative strain. These values are within the range of CLSI resistance breakpoint concentrations for each antibiotic as indicated in Table 2. The stains selected for this example method of operation include Gram-positive 1 ATCC 29213, Gram-positive 2 BAA-1026, Gram-negative 1 AR55 and Gram-negative 2 AR58 with cultures of OD₆₀₀=0.1, for simplicity.

Both Gram-positive strains were correctly classified using 2×(12 μg/mL oxacillin) in <120 min (97±45 min and 103±33 min). The Gram-negative strains were also correctly classified after increasing concentrations of imipenem from 2× to 20×(12 to 120 μg/mL imipenem). Surprisingly and unexpectedly, the analysis time for the imipenem susceptible and resistant strains were 146±40 min and 103±45 min, respectively. These phenotypic results were confirmed with results of standard procedure known to those skilled in the art and are included in Table 4 below, wherein READAS(S) susceptibility was defined by an ASIₙ≥0.4; (R) resistant was defined by an ASIₙ<0.4; analysis time shown in minutes. The standard procedure established susceptibility or resistance, MIC in μg/mL, and analysis time shown in hours.

TABLE 4

| | | | | | | | Standard |
|---|---|---|---|---|---|---|---|
| | | | | | | READAS | procedure |
| | | | | | | analysis | analysis |
| | | READAS | | Standard procedure | | time | time |
| Species | ASIn | Oxacillin | Imipenem | Oxacillin | Imipenem | (minutes) | (Hours) |
| Gram- | 0.55 ± 0.14^{b} | S/S | | 0.5 | | 97 ± 45 | 12.70 |
| positive | | | | | | | |
| 1 | | | | | | | |
| Gram- | −0.04 ± 0.19^{b} | R/R | | ≥4 | | 103 ± 33 | 10.02 |
| positive | | | | | | | |
| 2 | | | | | | | |
| Gram- | 0.1 ± 0.23^{c} | | R/R | | ≥16 | 146 ± 40 | 10.20 |
| negative | | | | | | | |
| 1 | | | | | | | |
| Gram- | 0.99 ± 0.01^{c} | | S/S | | ≤0.25 | 103 ± 45 | 10.45 |
| negative | | | | | | | |
| 2 | | | | | | | |

The READAS results in Table 4 indicate that the electrical current measurements can respond to PMS reduction significantly faster than standard procedures known to those skilled in the art. It is to be noted that READAS can reliably detect a current response of 1 nA, corresponding to a change in PMS concentration of 2.07 μM per second. Surprisingly and unexpectedly, in the example method of operations of the embodiments herein, a robust electrochemical current response with a high dynamic range was observed in less than 30 minutes that distinguished the response of antibiotic-sensitive and -resistant bacteria under antibiotic challenge, compared to inconclusive results from standard procedures. Herein, resistant, and susceptible strains of important nosocomial pathogens, including both Gram-positive and -negative organisms (Acinetobacter baumannii, Staphylococcus aureus, Escherichia coli, or Klebsiella pneumoniae) were distinguished and their susceptibility to four antibiotics with different mechanisms of actions (tobramycin, imipenem, oxacillin, and ciprofloxacin) was evaluated.

The four different antibiotics represent three fundamentally different mechanisms of activity. Beta-lactams, like imipenem and oxacillin, bind to the DD-transpeptidase of bacteria, which prevents cross-linking activity in the peptidoglycan portion of the cell membrane. When cross-linking is prevented and cells divide, this results in a faulty membrane and cell lysis (i.e., bactericidal activity). The aminoglycoside tobramycin binds to a site on the 30S and 50S ribosome, which prevents formation of the 70S complex and consequently, mRNA cannot be translated (i.e., bacteriostatic activity). The fluoroquinolone ciprofloxacin works by inhibiting the activity of DNA topoisomerase and DNA gyrase, thereby blocking DNA replication (bactericidal). Regardless of the antibiotic's bactericidal or bacteriostatic classification, susceptible strains exhibited reduced current when challenged with antibiotics compared to antibiotic-free controls.

In all the tested combinations of bacterial strains and antibiotics, the susceptible strains were consistent with reduced current under antibiotic challenge. The magnitude of current reduction, however, was different depending on the bacterial strain, cell density and antibiotic concentration. To achieve standard results, an initial cell density (OD₆₀₀=0.1) that exhibited a sufficient signal range and a response time (≥200 nA current and an analysis time <90 minutes) was selected. The antibiotic susceptibility index of 0.4, which is the percent reduction of current magnitude in the presence vs. absence of antibiotic (ASIₙ, where n is antibiotic concentration as a multiple of CLSI resistance breakpoint) consistently distinguished antibiotic-resistant and -susceptible strains. The criterion was validated using operator blinded tests using Gram-positive and -negative bacteria. Surprisingly and unexpectedly, READAS successfully classified antibiotic susceptible and resistant strains within 150 min compared to >10 hours using conventional antibiotic susceptibility tests.

### Example 3: A Multi-Well Electrochemical Reactor for Simultaneous Assessment of Multiple Combinations of Antibiotics and Bacteria Strains

The need to use an antibiotic concentration higher than the CLSI resistance breakpoint is expected because of the high initial cell density as used in the assay herein. Typical microdilution assays are inoculated with approximately 5×10⁵ CFU/mL, while READAS requires 6×10⁷ to 3×10⁸ CFU/mL to generate >30 nA current with a response time below 120 min. Importantly, the same OD₆₀₀ was suitable for all four bacterial species tested herein.

Antibiotic concentration was a more complicated parameter with some cases requiring concentrations up to 20-fold greater than CLSI resistance breakpoints. In order to determine the susceptibility or resistance of an antibiotic by including different concentrations of the antibiotic in a timely manner, a need exists for a single device for simultaneous tests. Another important aspect is the testing of uncharacterized bacteria. A number of antibiotics with different concentrations will have to be tested for in order to determine the antibiotic to which the uncharacterized bacteria is susceptible.

FIG. 14 illustrates a beneficial configuration to interrogate a multiplex of samples (aliquots) for pathogen susceptibility/resistance, as has been described throughout the four corners of the application, but now with a complexity that necessitates the processing of a vast amount of information so as to enable rapid assessment as has been generally described above for single container devices above. The multi-well electrochemical reactor, as generally referenced by the numeral 200 is configured with multiple (n) electrochemical reactors (e.g., configured as wells/titer plates, etc.) grouped together. Such a device with electrochemical reactors is often coupled with instrumentation a processer/computer-based system 201 to include interfaces, multiplexers, etc., as described above, for analysis and to aid in application of the methodologies disclosed herein. It to be noted that while FIG. 14, includes the controller and data system 201 as generally depicted as a laptop computer (also denoted with bi-directional arrows to depict communication with rest of system 200), it is to be emphasized that the operation of components within system 200 or any other embodiment disclosed herein can equally be enabled by a controller and data system of various circuitry of a known type. Such a control and data system 201 (computing devices) can thus be in the form of a desktop computer or a laptop computer as shown in FIG. 14, or can be implemented by any one of or a combination of general or special-purpose processors (digital signal processor (DSP)), firmware, software, graphical user interfaces (e.g., LabVIEW), and/or hardware circuitry to provide instrument control (e.g., AC and DC power), data analysis, etc., for the example configurations disclosed herein. It is also understood that the system 200 of FIG. 14 can be controlled remotely (e.g., from another room) and/or the information (e.g., data analysis) can be wirelessly communicated to remote servers via Bluetooth, infra-red, near field communication, WiFi, LiFi, and Ultra-wideband, etc. to include the cloud when desired for convenience of information gathering or external analysis.

It is also to be noted that in using such example computing devices, it is to also to be appreciated that as disclosed herein, the incorporated individual software modules, components, and routines may be a computer program, procedure, or process written as source code in C, C#, C++, Java, Python, and/or other suitable programming languages. The computer programs, procedures, or processes may be compiled into intermediate, object or machine code and presented for execution by any of the example suitable computing devices discussed above. Various implementations of the source, intermediate, and/or object code and associated data may be stored in one or more computer readable storage media that include read-only memory, random-access memory, magnetic disk storage media, optical storage media, flash memory devices, and/or other suitable media. A computer-readable medium, in accordance with aspects of the present invention, refers to media known and understood by those of ordinary skill in the art, which have encoded information provided in a form that can be read (i.e., scanned/sensed) by a machine/computer/processor and interpreted by the machine's/computer's/processor's hardware and/or software. It is also to be appreciated that as used herein, the term “computer readable storage medium” excludes propagated signals, per sc.

Turning to the beneficially system 200 as shown in FIG. 14, such a system 200 is configured with a multi-well base 202 with each well 203 (n wells, often up to 96 wells, more often even up to 384 wells), for example, being configured with an optimum reactor volume, well number, color, pattern, texture, material and surface coating, and such a multi-well plate can also be manufactured using 3D printing technology. Each well is designed to receive a desired solution configured with at least one of: a growth media, an inoculation (an antibiotic), a buffer solution, a control solution, a pathogen, etc., similar to the discussion above for FIG. 1A and FIG. 1B. A cover plate 204 is also shown with a configured n number of apertures (not shown) to insulate sets of electrodes from other sets of electrodes (e.g., the top of the cover plate can be fitted with an insulating material, for example, a nonconductive rubber) with the apertures designed to firmly hold multiple sets of electrodes 205 (only one set referenced for convenience) in place.

As a particular exemplary design, such electrodes are as before, two or more electrodes, often 3 sets of electrodes with the often 3 sets of electrodes (e.g., a working electrode, a counter electrode, and a reference electrode) being disposed within an inner region of each well so as to contact a solution having, for example, the pathogen and mediators, etc. In such a configuration, potentiostat measurements for three electrode configurations (or even two electrode measurement) for each well can be provided as aided by the processor similar to that as shown and described with respect to FIG. 1A and FIG. 1B but now as shown and described with respect to an often novel multiplexed potentiostat arrangement of FIG. 14. Such a measurement process/system cannot be done without the aid of the processor 201 as the complexity of analysis would be too time consuming otherwise. In such a novel multiplexed system, the configurations for FIG. 14 include an array of electrodes 205 configured with dedicated circuits embedded in plate 206 (control op-amps, converters, etc.) for each electrode. More often, the system of FIG. 14 can be configured as a single-channel system with an interface 211 (e.g., a multiplexer, as stated above) that isolates signals from specific electrodes 205 so as to enable measurements from two or three electrode configurations, often potentiostat-like measurements of cells 203 without a cumbersome number of dedicated channels.

The plate 206 as shown in FIG. 14, with circuitry embedded (not shown) is thereafter operably linked to a computer/control and data system/processor 201, as was described above. While such a plate is depicted in a top configuration, as shown in FIG. 14, it is to be emphasized that the plate 206 can also be arranged in a bottom-type of configuration (not shown for simplicity) so as to enable shortening of the electrode structure and for case of operation. However, it is also to be noted that for any plate 206 type of configuration disclosed herein, electrodes configured to intercept solutions (e.g., a control solution, solutions having at least one of a pathogen, a drug (antibiotic), an electron transfer mediator, a growth media, a pathogen, etc.) and thus receive induced currents are generally not wholly embedded in the individual wells. For example, from any configuration, they can be arranged as suspended electrodes with a portion of the tips of the electrodes introduced in the solution of each well. Such an arrangement is beneficial in maintaining the rate of electron transfer and thus minimize signal perturbations by not allowing material from the inoculated cultures to settle onto the electrodes and hamper the process (signals) in any way. As an additional embodiment, Electrodes can also be embedded within the plate 206 itself as a part of a disposable plate. In this case, two or three electrodes are located within each well (embedded as a part of the walls of each individual well 203).

While the foregoing invention is described with respect to the specific examples, it is to be understood that the scope of the invention is not limited to these specific examples. Since other modifications and changes varied to fit particular operating requirements and environments will be apparent to those skilled in the art, the invention is not considered limited to the example(s) chosen for purposes of disclosure and covers all changes and modifications which do not constitute departures from the true spirit and scope of this invention.

I/We claim:

1. A multiplexed electrochemical antibiotic susceptibility method, comprising:
disposing a non-inoculated solution in one or more sample wells arranged in an array, wherein the non-inoculated solution in the one or more sample wells is configured with at least one of an electron transfer mediator, a growth culture, and a pathogen;
measuring a multiplexed number of current control signal responses of the non-inoculated solution disposed in the one or more sample wells of the array;
inoculating a set number of solutions in the one or more sample wells with an antibiotic, wherein the inoculating step includes a range of concentrations of up to about 24 times a breakpoint of the antibiotic;
measuring a multiplexed number of inoculated current response signals from the inoculated set number of solutions in the one or more sample wells of the array; and
analyzing with a computer control and data system/processor, the multiplexed number of current control signal responses and the multiplexed number of inoculated current response signals over the range of concentrations of up to about 24 times a breakpoint of the antibiotic to provide a susceptibility index assessment indicative of the susceptibility or resistance of the pathogen to the antibiotic.

2. The multiplexed electrochemical antibiotic susceptibility method of claim 1, wherein the electron transfer mediator is at least one of an exogenous mediator and an endogenous mediator.

3. The multiplexed electrochemical antibiotic susceptibility method of claim 1, wherein the susceptibility index assessment includes measuring a time frame at an inflection point of an exponential growth of a number of current response and a saturated number of current responses.

4. The multiplexed electrochemical antibiotic susceptibility method of claim 3, wherein when the susceptibility index assessment using the inflection point of an exponential growth of a number of current response and the saturated number of current responses is determined in the analyzing step to be a value greater than or equal to a threshold, the pathogen is determined to be susceptible to the antibiotic, and wherein when the susceptibility index assessment indicates a value of less than the threshold, the pathogen is determined by the analyzing step to be resistant to the antibiotic.

5. The multiplexed electrochemical antibiotic susceptibility method of claim 4, wherein the threshold is 0.4.

6. The multiplexed electrochemical antibiotic susceptibility method of claim 1, wherein the electron transfer mediator is at least one of an exogenous mediator and an endogenous mediator.

7. A multiplexed electrochemical drug susceptibility system, comprising:
an array of sample containers, wherein one or more sample containers of the array of sample containers are each configured with an interior volume configured to hold a solution that further comprises at least one of an electron transfer mediator, a drug, a growth culture, and a pathogen;
a plurality of sets of two or more electrodes, wherein each of a set of the plurality of sets of two or more electrodes are coupled to the solution of the one or more sample containers;
an interface configured to send and isolate a multiplexed set of control signals and a multiplexed set of current response signals from the plurality of sets of two or more electrodes; and
a computer control and data system/processor coupled to the interface so as to interrogate the multiplexed set of control signals and the current response signals for a susceptibility index assessment of the pathogen.

8. The multiplexed electrochemical drug susceptibility system of claim 7, wherein each of the set of the plurality of sets of two or more electrodes are further configured with a working electrode to form a portion of one or more potentiostats.

9. The electrochemical antibiotic susceptibility system of claim 7, wherein the system is configured with a dedicated circuit for each electrode of the plurality of sets of two or more electrodes.

10. The multiplexed electrochemical drug susceptibility system of claim 7, wherein the array of sample containers is configured as a multi-well plate configured with up to at least 384 wells.

11. The multiplexed electrochemical drug susceptibility system of claim 7, wherein the one or more of the sample containers can be configured with an increased concentration of up to about 24 times a breakpoint of the drug.

12. The multiplexed electrochemical drug susceptibility system of claim 7, wherein the drug is an antibiotic.

13. The multiplexed electrochemical drug susceptibility system of claim 7, wherein the electron transfer mediator is at least one of an exogenous mediator or an endogenous mediator.

14. The multiplexed electrochemical antibiotic susceptibility method of claim 7, wherein when one or more computer control and data system/processor susceptibility index assessments are output as one or more measured values greater than or equal to a threshold, the pathogen is determined to be susceptible to the antibiotic, and wherein when the one or more measured values are output as values less than the threshold, the pathogen is resistant to the antibiotic.

15. The multiplexed electrochemical antibiotic susceptibility method of claim 14, wherein the threshold is 0.4.
